(19) United States
(12) Patent Application Publication
(10) Pub. No.: US 2008/0280914 A1
(43) Pub. Date: Nov. 13, 2008

(54) Intravenous Formulations of Pde Inhibitors

(75) Inventors: Peter Serno, Bergisch Gladbach (DE); Helmut Haning, Wuppertal (DE); Frank Reetz, Koln (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG, LEVERKUSEN (DE)

(21) Appl. No.: 11/795,361

(22) PCT Filed: Jan. 5, 2006
(86) PCT No.: PCT/EP2006/000045

§ 371 Date: Jun. 17, 2008

(30) Foreign Application Priority Data
Jan. 15, 2005 (DE) 10 2005 001 989.7

(51) Int. Cl. A61K 31/4985; A61K 31/519; A61P 1/00; A61P 13/12; A61P 15/00; A61P 9/00; A61P 27/16

(52) U.S. Cl. 514/250; 514/252.16

(57) ABSTRACT

The invention relates to a novel pharmaceutical dosage form of PDE 5 inhibitors for intravenous administration and the use thereof for the treatment of diseases.

The invention relates to novel applications of intravenous administration forms of PDE inhibitors and to novel pharmaceutical formulations therefor.

PDE, especially PDE 5 inhibitors are known as potent active pharmaceutical ingredients and are employed for the treatment of diseases. Thus, for example, the compound vardenafil with the systematic name {2-ethoxy-5-[4-ethyl-1-piperazinyl)sulfonyl]phenyl}-5-methyl-7-propylimidazol[5,1-ƒ]triazin-4(3H)one and its physiologically acceptable salts is described for example in WO99/24433. Other PDE 5 inhibitors are

sildenafil, tadalafil,

DA8159: enantiomers of 5-[2-propyloxy-5-(1-methyl-2-pyrrolidinylethylamido-sulfonyl)phenyl]-1-methyl-3-propyl-1,6-dihydro-7H-pyrazolo(4,3-d)pyrimidine-7-one as described in WO 2001098304,

TA 1790: avanafil, (S)-2-(2-hydroxymethyl-1-pyrrolidinyl)-4-(3-chloro-4-methoxy-benzylamino)-5-[(2-pyrimidinylmethyl)carbamoyl]pyrimidine

EMD-221829: 4-{4-[(3-chloro-4-methoxybenzyl)amino][1]benzothieno[2,3-d]-pyrimidin-2-yl}cyclohexanecarboxyl acid ethanolamine salt

QAD-171A as described in WO00177110,

PT131 and

ABT724: 2-[(4-pyridin-2-ylpiperazin-1-yl)methyl]-1H-benzimidazole.

Vardenafil leads, as do sildenafil and tadalafil, through the PDE 5 inhibition to an inhibition of the intracellular degradation of cGMP. As a consequence, NO activation results in elevated intracellular cGMP levels. The mechanism has been described to date for the treatment of erectile dysfunction and for the treatment and prophylaxis of further disorders such as hypertension, neuronal hypertension, stable and unstable angina, peripheral and cardiac vascular disorders, of arrhythmias, for the treatment of thromboembolic disorders and ischemias such as myocardial infarction, stroke, transient ischemic attacks, angina pectoris, peripheral blood flow impairments, for preventing restenoses following thrombolysis therapy, percutaneous transluminal angioplasty (PTA), percutaneous transluminal coronary angioplasty (PTCA) and bypass.

Besides these known uses, it has now been found that PDE 5 inhibitors can be employed for the treatment of numerous further disorders for which the possibility of therapy by PDE 5 inhibitors have not to date been suspected, especially when the particular compound (or compounds) is (or are) supplied intravenously.

Differential expression of phosphodiesterases in different cells, tissues and organs, as well as differential subcellular localization of these enzymes, make it possible, especially when the PDE inhibitors are supplied intravenously, for the various processes regulated by cGMP to be addressed selectively. The preparations of the invention are therefore suitable for the prophylaxis and/or treatment of disorders in which an increase in the cGMP concentration is beneficial, i.e. disorders connected with cGMP-regulated processes (usually referred to simply as cGMP-related diseases). The PDE 5 inhibitors moreover enhance the effect of substances such as, for example, EDRF (endothelium derived relaxing factor), ANP (atrial natriuretic peptide), of nitrate vasodilators and all substances which increase the cGMP concentration in a different way than phosphodiesterase inhibitors.

Specifically, PDE 5 inhibitors now make it possible after administration in the form of the infusion formulations of the invention also to treat cardiovascular disorders. Examples are: hypertension, heart failure, pulmonary hypertension, nitrate-induced tolerance, neuronal hypertension, stable and unstable angina, peripheral and cardiac vascular disorders, achieving or improving a preconditioning effect, cardiac ischemia, acute myocardial infarction, reperfusion damage, specifically following a myocardial infarction, arrhythmias, thromboembolic disorders and ischemias such as myocardial infarction, coronary heart disease, stroke, transient and ischemic attacks, angina pectoris, peripheral blood flow impairments, Raynaud's syndrome and intermittent claudication. They are further suitable for preventing restenoses following thrombolysis therapy, percutaneous transluminal angioplasty (PTA), percutaneous transluminal coronary angioplasties (PTCA) and bypass.

The infusion formulations of the invention comprising PDE 5 inhibitors can further be employed for the treatment of disorders of the urogenital system such as prostate hypertrophy, incontinence, bladder disorders, erectile dysfunction, priapism, Peyronie's disease, premature labor, premature ejaculation, male infertility, inadequate sperm motility, dysmenorrhea, polycystic ovary syndrome, incontinence (e.g. urge incontinence), acute and chronic renal failure, renal syndrome, glomerular disease, nephritis, tubulointestinal disorders, glomuleropathy, female infertility, female sexual dysfunction and female sexual arousal impairment. Use in reproductive medicine is also possible, for example to promote the growth and improve the survival of oocytes, zygotes, embryos or fetuses, for increasing the weight of premature infants, for increasing milk production in mammals, specifically in humans, for premature labor and pre-eclampsia.

A further area of use is the treatment and/or prophylaxis of impairments of perception, of concentration, of learning and/or memory, especially if the impairment is a consequence of dementia. The formulations used according to the invention are particularly suitable for improving perception, concentration, learning, or memory following cognitive impairments like those occurring in particular in situations/diseases/syndromes such as mild cognitive impairment, age-associated learning and memory impairments, age-associated memory loss, vascular dementia, craniocerebral trauma, stroke, dementia occurring after strokes (“post stroke dementia”) and post-traumatic craniocerebral trauma. Use is also possible for concentration impairments in children with learning and memory problems, Alzheimer's disease, vascular dementia, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, multiple sclerosis, thalamic degeneration, Creutzfeld-Jacob dementia, HIV dementia, schizophrenia with dementia or Korsakoff's psychosis, treatment of depression, amnesia, disturbances of consciousness, autism, speech impairments, Lennox syndrome and epilepsy.

Use of intravenous formulations of the invention comprising PDE 5 inhibitors is additionally possible for the treatment or prophylaxis of disorders of the eye such as glaucoma, especially acute glaucoma, central retinal or posterior cilliary arterial occlusion, central retinal venous occlusion, optic neuropathy such as anterior ischemic optic neuropathy and glaucomatous optic neuropathy, and of macular degeneration.

Further areas of use are diabetes, insulin resistance, hyperglycemia, diabetic gastroparesis, diabetic nephropathy, diabetic neuropathy, diabetic retinopathy, diabetic gangrene, diabetic glomerulosclerosis, diabetic dermopathy, diabetic arthropathy, diabetic dermatopathy and diabetic cataract.

The intravenous formulations of the invention comprising PDE 5 inhibitors are also suitable for the treatment of the following disorders: impairments of the peristalsis of stomach and esophagus, hepatic disorders such as, for example, cirrhosis of the liver, portal hypertension, pancreatitis, inflammatory bowel disease (such as, for example, Crohn's disease and ulcerative colitis), impairments of gastric motility, also for supporting and promoting liver regeneration following surgical resection of the liver or liver cancer and for inhibiting the contraction of the esophageal muscles (e.g. nutcracker esophagus, spastic esophageal disorder).

The formulations of the invention can additionally be employed for the prophylaxis and/or treatment of: osteoporosis, psoriasis, cancer, cystic fibrosis, alopecia, pain, tinnitus, sudden loss of hearing, COPD, asthma, bronchitis and allergic rhinitis, fibrotic disorders, arteriosclerosis, leukemia (e.g. chronic lymphocytic leukemia), platelet adhesion and aggregation associated with renal ischemia, achalasia, hypertensive LES, lupus, scleroderma, hair loss or loss of hair, multiple sclerosis and rheumatoid arthritis, allergy, osteoporosis, autoimmune diseases, cachexia, hyperlipidemia and dyslipidemia, and migraine.

Formulations which can be administered intravenously of PDE 5 inhibitors, especially vardenafil, represent a further aspect of the invention.

Solutions of vardenafil and its physiologically acceptable salts are described in WO99/24433. It is intended for their preparation that the therapeutically active compound be present in a concentration of from 0.5 to 90% by weight of the complete mixture. However, it has emerged that the low solubility in water and instability of vardenafil in numerous organic solvents stand in the way of a conventional formulation of vardenafil to give preparations which can be used intravenously. In addition, the stated concentration of the active ingredient in the formulation permits only a rapid intravenous supply of the active ingredient, for example as bolus injection or infusion at a very low infusion rate.

According to the present invention, a formulation of PDE 5 inhibitors such as vardenafil which can be used intravenously and is easy to handle and well tolerated can be obtained when 0.0004 to 0.1% (m/v) of the PDE inhibitor are dissolved in the form of the free base or of a salt in an aqueous solvent. Solutions particularly preferred in this connection are those which comprise an acid in addition to the PDE inhibitor. A molar ratio of amounts of from 1:0.9 to 1:2.0 (PDE inhibitor: acid) is particularly preferred in this connection. When PDE inhibitors are employed in the form of a salt, the amount of acid to be added is reduced by the amount already employed for the salt formation. In the case of polyprotic acids, depending on the acid strength of the respective dissociation stage, the stated amount of acid may where appropriate be divided by the number of protons released per molecule of acid.

Compared with previously disclosed formulations for example of vardenafil, the infusion solutions of the invention have the advantage of being well tolerated after parenteral administration, a virtually immediate buildup of effective plasma concentrations, easy controllability of the supply of drug because the infusion rate can be reduced if unwanted side effects occur. A particular advantage is represented by the very high bioavailability after administration of the preparations of the invention, which is surprisingly 6 to 7 times higher than that of a tablet given orally.

Specifically, to prepare the solutions of the invention, the PDE 5 inhibitor is dissolved in amorphous, crystalline or solvent-containing form in an aqueous solvent. This is done by adding one or more acids thereto. Examples of suitable acids are: acetic acid, adipic acid, ascorbic acid, aspartic acid, benzenesulfonic acid, benzoic acid, citric acid, ethanesulfonic acid, 2-hydroxyethanesulfonic acid, fumaric acid, glucoheptoic acid, gluconic acid, glucuronic acid, glutamic acid, hydrochloric acid, lactic acid, lactobionic acid, maleic acid, malic acid, malonic acid, methane-sulfonic acid, naphthalenesulfonic acid, naphthalenedisulfonic acid, nitric acid, phosphoric acid, succinic acid, sulfuric acid, tartaric acid, toluenesulfonic acid, mono- or diesters of orthophosphoric acid such as, for example, glycerol phosphate. In the case of polyprotic acids, it is also possible to employ their acidic salts such as, for example, sodium bisulfate or sodium dihydrogen phosphate.

It is further possible to add to the formulations of the invention an isotonicity agent, for example sodium chloride, glucose, fructose, mannitol, sorbitol, glycerol, acetate buffer, citrate buffer, phosphate buffer or lactate buffer or amino acids.

The pH of the preparations can be adjusted with one of said acids or, if the pH is already too acidic, with a base such as sodium hydroxide, trometamol, arginine or lysine. A pH range preferred for the formulations of the invention is from 3 to 7.

To improve the solubility, it is also possible to add organic solvents which can be administered parenterally, such as ethanol, propylene glycol or polyethylene glycol, surfactants or polymers such as polyvinylpyrrolidone, polysorbate, poloxamer, Cremophor, Solutol HS 15, phospholipids and native or substituted cyclodextrins.

The formulations of the invention are dispensed into known containers for parenteral administration, for example into injection vials or infusion bottles made of glass with stoppers, into flexibags or into other large- or small-volume containers made of plastics, into prefilled syringes or carpules. Dispensing into plastics containers is also possible by the blow-fill-seal process.

The preparations of the invention are prepared for example by dissolving vardenafil or a vardenafil salt together with acid, isotonicity agents and, where appropriate, further excipients in the solvent (usually water). Adjustment of the pH is followed by making up to the total amount employed with water, sterilization by filtration through 0.2 μm filter membranes and dispensing. Although an entirely aseptic preparation process or a lyophilization of the formulations of the invention are possible, generally sterilization of the dispensed solution in the final container is preferred, for example at 121° C. for 15 minutes. If packagings which do not withstand this temperature without harm are used, however, aseptic preparation is possible, without or with subsequent thermal treatment, possibly at temperatures below 121° C.

Concentrates represent a particular embodiment of the invention. In order to avoid the costly transport and storage of large-volume containers, firstly a concentrated solution of vardenafil is prepared and distributed. The infusion solution of the invention is then prepared by the user, for example by adding the concentrate solution to a standard infusion or by continuous dilution of the concentrate via a Y piece.

The infusion solutions of the invention can be administered intravenously in various ways depending on the dose of active ingredient, the concentration of active ingredient and the area of use. Administration as bolus injection, administration in the form of a gravity drip infusion or pumping through an infusion tubing pump or infusion syringe driver are possible. The infusions are generally administered into peripheral veins, but central venous or, in special cases, also arterial administration is possible for intensive care patients.

Comparative examples 1-2 detailed below represent preparations not according to the invention which are detailed to illustrate the advance achieved by the formulations of the invention.

### EXAMPLE 1 (COMPARATIVE)

Preparation not according to the invention, active ingredient concentration 0.005 mg/ml

| | | |
|---|---|---|
| Vardenafil dihydrate | 0.005 | g |
| Sodium chloride | 9.00 | g |
| Water for injections | 991 | g |

The solution contains considerable amounts of undissolved active ingredient and is unsuitable for intravenous infusion.

### EXAMPLE 2 (COMPARATIVE)

Preparation not according to the invention with 70% polyethylene glycol 400

| | | |
|---|---|---|
| Vardenafil dihydrate | 0.50 | g |
| Polyethylene glycol 400 | 700 | g |
| Water for injections | 299.5 | g |

The solution is unstable. 6.5% vardenafil N-oxide is formed even on preparation of the solution. The content thereof increases to 11% following heat sterilization of the solution.

### EXAMPLE 3

Stability and biological demonstration of the good tolerability and exceptional bioavailability of formulations of the invention

| | | |
|---|---|---|
| Vardenafil hydrochloride trihydrate | 0.119 | g |
| Sodium chloride | 9.00 | g |
| 20% lactic acid solution | 5.00 | g |
| 2M sodium hydroxide solution ad pH | 4.00 to 10 | g |
| Water for injections up to the total amount employed | 1005.1 | g |

20 ml of this solution (equivalent to 20 mg of vardenafil free base) was administered to each of 12 subjects in a crossover test compared with a tablet containing 11.85 mg of vardenafil HCl trihydrate (equivalent to 10 mg of vardenafil free base). For this purpose, the solution was continuously infused over about 1 hour. The tolerability of the infusion was good. All observed side effects were generally mild to moderate and reversible after completion of the study. Only in one subject was a mild reaction observed at the injection site. The bioavailability AUC determined from the plasma concentrations was 35.4 μg*h/l (geometric mean) for the infusion formulation of the invention and 25.7 μg*h/l (geometric mean) for the tablet. Taking account of the dosage administered, this reveals a bioavailability for the infusion solution of 689% of the tablet.

The stability of this solution over 13 weeks at 6° C., 25° C. and 40° C. was also investigated. The vardenafil content was initially 0.100 mg/ml and was 0.099 mg/ml at the end of storage under all conditions. The total of all the degradation products was initially undetectable (<0.02%); likewise undetectable (<0.02%) after 13 weeks at 6° C., <0.1% after 13 weeks at 25° C. and 0.1% after 13 weeks at 40° C. The values show the excellent stability of the formulations of the invention.

### EXAMPLE 4

0.268 kg of vardenafil dihydrate, 61.5 g of methanesulfonic acid and 25.9 kg of mannitol are dissolved in 174.7 kg of water for injections under aseptic conditions. The solution is sterilized by filtration and dispensed in 1.6 g portions into injection vials. The solution is lyophilized in the injection vials, stoppered and crimp-capped. The product is distributed in this form. The user then reconstitutes the lyophilizate and transfers it into 100 ml of 5% strength glucose solution, which then has the following composition on use:

| | | |
|---|---|---|
| Vardenafil dihydrate (equivalent to 2.00 mg of vardenafil) | 2.15 | mg |
| Methanesulfonic acid | 0.492 | mg |
| Mannitol | 200 | mg |
| Glucose | 5.00 | g |
| Water for injections | 96.6 | g |

### EXAMPLE 5

107.4 mg of vardenafil dihydrate, 27.7 mg of tartaric acid and 9 g of sodium chloride are dissolved in one liter of water for injections. The solution is sterilized by filtration, dispensed in 2 ml quantities into prefilled syringes and sterilized. Each prefilled syringe contains 0.2 mg of vardenafil.

### EXAMPLE 6

859 mg of vardenafil dihydrate and 452 mg of citric acid are dissolved in 900 ml of water for injections. The volume is then made up to 1 liter with water for injections. The solution is sterilized by filtration through 0.2 μm filter, dispensed in amounts of 5.0 ml into vials and heat-sterilized at 121° C. for 15 minutes. The solution is added before use to 500 ml of 5% glucose solution and infused slowly.

### EXAMPLE 7

5.72 mg of vardenafil dimesilate monohydrate is added to 1000 ml of physiological saline solution. The solution is sterilized by filtration and introduced in amounts of 250 ml under aseptic conditions into infusion bottles. Each infusion bottle contains 1 mg of vardenafil.

### EXAMPLE 8

10 g of sildenafil, 500 g of 0.1M hydrochloric acid and 5 kg of glucose are dissolved in 96.1 kg of water for injections, sterilized by filtration and introduced in amounts of 100 ml under aseptic conditions into infusion bottles.

### EXAMPLE 9

0.005 kg of tadalafil is dissolved in 30 kg of polyethylene glycol 400 and 30 kg of 96% ethanol. The volume is made up to 200 liters with water for injections. The solution is sterilized by filtration and dispensed in amounts of 100 ml aseptically into infusion bottles.

1. An intravenous formulation comprising at least one PDE 5 inhibitor or a salt thereof.

2. The intravenous formulation as claimed in claim 1, comprising vardenafil and/or a salt thereof, tadalafil and/or a salt thereof or sildenafil and/or a salt thereof as said PDE 5 inhibitor.

3. The intravenous formulation as claimed in claim 1, comprising vardenafil and/or a salt of vardenafil as said PDE 5 inhibitor.

4. A method for the treatment of portal hypertension, stroke, craniocerebral trauma, premature labor, acute renal failure, acute glaucoma, pancreatitis, sudden loss of hearing, tinnitus, achalasia or spastic esophageal disorder comprising administering an intravenous formulation which comprises at least one PDE 5 inhibitor.

5. A method for the treatment of portal hypertension, stroke, craniocerebral trauma, premature labor, acute renal failure, acute glaucoma, pancreatitis, sudden loss of hearing, tinnitus, achalasia or spastic esophageal disorder comprising administering an intravenous formulation which comprises vardenafil, sildenafil or tadalafil.

6. The intravenous formulation as claimed in any of claims 1 to 3, comprising vardenafil in a concentration of from 0.005 to 0. 1% by weight as said PDE 5 inhibitor.

7. The intravenous formulation as claimed in any of claims 1 to 3, comprising vardenafil and acid in the molar ratio of 1:0.9-2.0 as said PDE 5 inhibitor.

8. A method for the differential treatment and selective addressing of the various processes regulated by cGMP comprising administering an intravenous formulation which comprises at least one PDE 5 inhibitor.
